(19) United States
(12) Patent Application Publication
(10) Pub. No.: US 2009/0254159 A1
(43) Pub. Date: Oct. 8, 2009

(54) Method and Device for Controlling the Temperature of Local Regions of a Patient's Body

(75) Inventor: Johan Stormby, Limhamn (SE)

Correspondence Address: HARNESS, DICKEY & PIERCE, P.L.C., P.O. BOX 8910, RESTON, VA, 20195, US

(73) Assignee: PROCELLA AB, Malmo (SE)

(21) Appl. No.: 12/086,060

(22) PCT Filed: Dec. 6, 2006
(86) PCT No.: PCT/SE2006/001383

§ 371 Date: Jun. 11, 2009

(30) Foreign Application Priority Data
Dec. 6, 2005 (SE) 0502679-4

(51) Int. Cl. A61F 7/00

(52) U.S. Cl. 607/104

(57) ABSTRACT

Method and device are disclosed for controlling the temperature of local portions of a patient's body. The temperature of regions including the nails of fingers and or toes is controlled to be kept at a predetermined low level when the patient is undergoing chemotherapy. In at least one embodiment, the device for controlling the temperature includes a heat insulating body forming cavities for receiving therein the patient's fingers and/or toes, and passages in said body for flowing a coolant through regions in heat exchanging relation to said cavities receiving the nails of the patient's fingers or toes when inserted into the cavities.

### BACKGROUND OF THE INVENTION

A human being undergoing treatment with cytostatica (chemotherapy) or a similar treatment often suffers of loss of hair. By maintaining during the treatment a low temperature on the hairy region, usually the scalp, such loss can be eliminated or substantially reduced.

U.S. Pat. No. 6,156,059 discloses a head cooler for this purpose comprising a refrigeration unit and a head cover to be placed over a patient's scalp for enclosing the patient's head and neck, flow passages for circulating a fluid coolant supplied by the refrigeration unit, through the device being provided internally of the cover which forms an inside heat exchange surface to be applied against the scalp. A cooling system of this kind for enduring chemotherapy treatment is marketed under the trade mark DigniCap by Dignitana AB, a Swedish company of Malmö, Sweden. The refrigeration unit included in this cooling system has connections for connecting to the unit two separately continuously temperature controlled cooling sections of the cap.

It has been found that also nail changes are a common side effect of systemic chemotherapy, including discoloration, ridging or pitting of the nails and, as the most severe toxic effect in this respect, partial or complete loss of nails or pain in the nail beds of the patient's hands or feet.

U.S. Pat. No. 6,141,801 and WO 99/470 11 describe a glove fitted to a human hand and having a pocket on the palm side or back-hand side of the glove receiving a thermal gel-filled pack adapted for repeated heating and cooling, uncontrolled transmission of heat or cold taking place from the gel-filled pack to other parts of the hand for therapeutic or other purposes.

### BRIEF DESCRIPTION OF THE INVENTION

The present invention relates to method and device for controlling the temperature of defined local regions of a patient's body.

The object of the invention is to alleviate or eliminate the inconvenience or pain suffered in the nails of hands and/or feet by a patient undergoing chemotherapy by allocation of the cooling effect to the site or sites from which the pain or other inconvenience emanates i. e. particularly the nail bed on hands and/or feet, and by accurately controlled local cooling of said site or sites in order to effectively utilize the cooling effect for therapeutic purposes.

This object is achieved by the method defined in claim 1.

A device for controlling the temperature of defined local portions of the body of a patient undergoing chemotherapy according to claim 2 is characterized by a heat insulating body forming cavities for receiving therein the patient's fingers and/or toes, and by passages internally of said body for flowing a fluid coolant through regions in heat exchanging relation to said cavities receiving the nails of the patient's fingers or toes when inserted into the cavities

Further features of the invention are defined in the dependent claims.

### BRIEF DESCRIPTION OF THE DRAWINGS

In the accompanying drawings which disclose illustrative embodiments of the method and device according to the invention,

FIG. 1 is a horizontal cross sectional view of a device according to the invention for therapeutic treatment of the hand nails,

FIG. 2 is a vertical cross sectional of the device in FIG. 1,

FIG. 3 is vertical cross sectional view of a device of the invention for therapeutic treatment of the foot nails, and

FIG. 4 is a horizontal sectional view of the device in FIG. 3.

### DETAILED DESCRIPTION OF THE ILLUSTRATIVE EMBODIMENTS

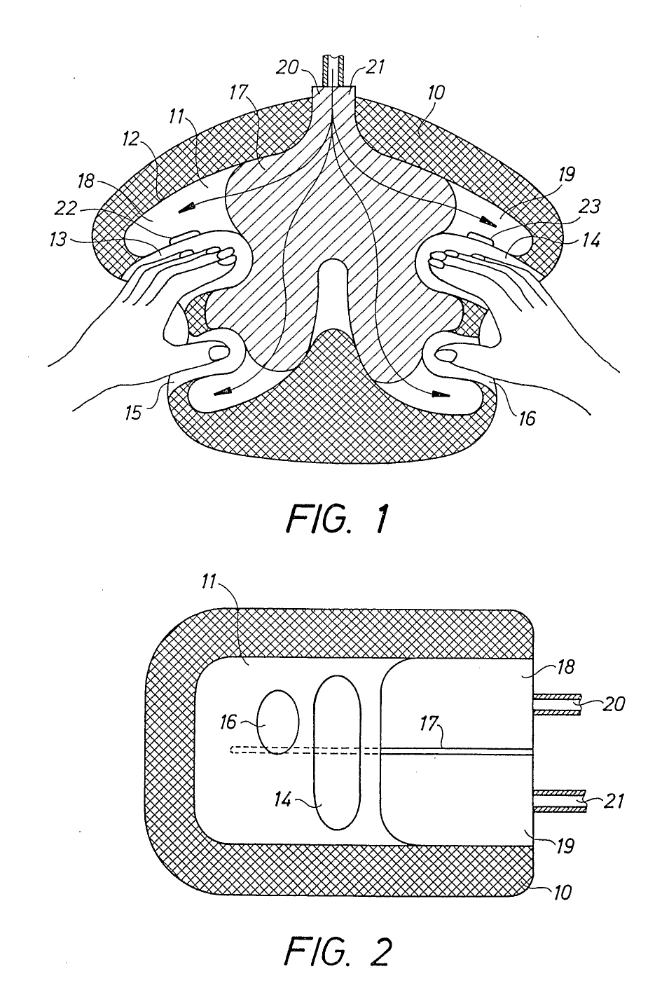

The device shown in FIGS. 1 and 2 comprises a body 10 which in its entirety is made of a heat insulating material or includes an inner and an outer shell with a heat insulating material therebetween. An interior space 11 in body 10 is provided with a lining 12 of a fluid impermeable thin and soft material, preferably silicone, the thickness of lining 12 being of the order of 1 or 2 mm. Lining 12 forms two cavities 13 and 14, which open in the outer surface of the body and are adapted to receive the fingers on the left hand and the right hand, respectively, of a patient who shall undergo therapeutic treatment by means of the device, and two cavities 15 and 16 adapted to receive the thumb on the patient's left hand and right hand, respectively. Also these cavities open in the outside surface of the body so that the fingers and the thumbs, respectively, can be inserted into the cavities from the outside of the body. It is the intention that body 10 during treatment of a patient shall be located in the patient's lap with the fingers and thumbs inserted in the associated cavities so that the patient can take a comfortable and relaxed position during the treatment.

The interior space 11 in body 10 is divided by a partition 17 into two compartments 18 and 19 on the upper and lower side of the partition, respectively, compartment 18 being connected to a hose or tube 20 for supply of fluid coolant and compartment 19 being connected to a hose or tube 21 for withdrawal of the coolant from the interior of body 10. Thus, the fluid coolant is forced to circulate through the interior space 11 of body 10 in heat exchanging contact with the heat conducting material 12 cavities 13 to 16 being cooled by the circulating fluid coolant which is delivered from a refrigerating unit and then after passage through the interior of body 10 is returned to the refrigerating unit.

Temperature sensors 22 and 23 are mounted to the outside wall of cavities 13 and 14 and the temperature and flow of the coolant is controlled in dependence of the temperature sensed by these sensors in order to maintain in cavities 13 to 16 the temperature necessary in order to avoid pain and other inconvenience at the nails of the fingers of the patient undergoing therapeutic treatment by means of the device. Further temperature sensors can be provided in the interior space 11 of body 10 at suitable locations therein.

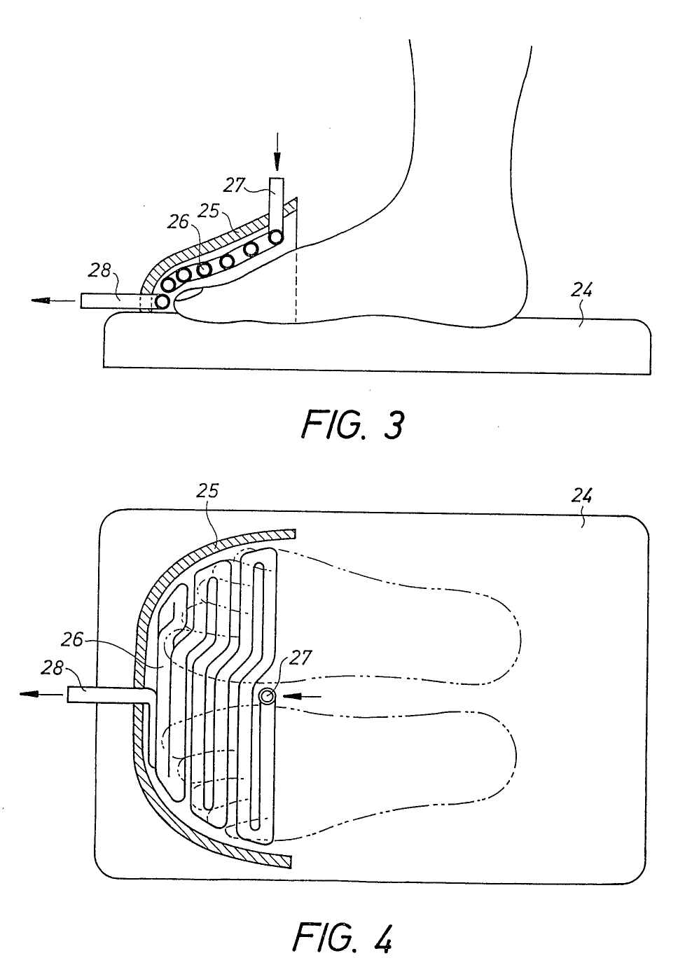

FIGS. 3 and 4 disclose a device for treatment of a patient's toes in order to eliminate the severe side effects of chemotherapy. A foot plate 24 of a soft material or alternatively with a soft padding on a rigid substrate is provided with a cover 25 of a heat insulating material. Cover 25 is open at one side thereof so that the toes of a foot can be inserted therein by slipping the foot along the foot plate towards the open side of the cover. Inside the cover there is mounted at the lower side thereof a serpentine shaped tube or hose coil 26 for circulating a fluid coolant, having an inlet 27 and an outlet 28 to be connected to a refrigeration unit in order to maintain inside the cover in the region of the toes on the foot inserted under the cover a predetermined low temperature. One or more temperature sensors can be mounted inside the cover for continuous control of the temperature in said region. If the refrigeration unit is of the kind mentioned above (DigniCap) with connections for two separate cooling sections, two devices with separate temperature control, one for cooling the finger and thumb nails and one for cooling the toe nails, can be connected simultaneously to the refrigeration unit.

1. A method for controlling the temperature of local portions of a patient's body, comprising:
controlling the temperature of regions, including nails of at least one of fingers and of the patient's body, to keep them at a predetermined low level when the patient is undergoing chemotherapy.

2. A device for controlling the temperature of local portions of a body of a patient undergoing chemotherapy, comprising:
a heat insulating body forming cavities opening in an outside surface of the body for receiving therein at least one of the patient's fingers and toes; and
passages in said heat insulating body for flowing a coolant through regions in heat exchanging relation to said cavities receiving the nails of at least one of the patient's fingers and toes when inserted into the cavities.

3. The device of claim 2 wherein the body forms an interior space for flowing fluid coolant through the body.

4. The device of claim 3 wherein the interior space is lined with a fluid impermeable material.

5. The device of claim 4 wherein the lining defines said cavities.

6. The device of claim 2 wherein at least one of said cavities is provided with an inside hose or tube system for circulating fluid coolant through a region of said at least one cavity.

7. The device of claim 6 wherein the cavity is formed by a cover for receiving the front portion of a foot.

8. The device of claim 7 wherein the cover forms a side opening for insertion of the foot.
